(19) United States
(12) Patent Application Publication
(10) Pub. No.: US 2024/0266023 A1
(43) Pub. Date: Aug. 8, 2024

(54) NUTRITIONAL COMPOSITIONS AND METHODS FOR MITIGATING INADEQUATE NUTRITIONAL INTAKE OF A TIME-RESTRICTED FEEDING REGIMEN

(71) Applicant: SOCIETE DES PRODUITS NESTLE S.A., Vevey (CH)

(72) Inventors: PALOMA ELORTEGUI PASCUAL, Epalinges (CH); ALISON L. ELDRIDGE, Pully (CH); ERIC ANTOINE SCUCCIMARRA, Lausanne (CH); FABIO MAINARDI, Lausanne (CH)

(21) Appl. No.: 18/569,277

(22) PCT Filed: Jun. 15, 2022
(86) PCT No.: PCT/EP2022/066251

§ 371 Date: Dec. 12, 2023

(30) Foreign Application Priority Data
Jun. 15, 2021 (EP) 21179451.6

(51) Int. Cl. G16H 20/60

(52) U.S. Cl. CPC G16H 20/60

(57) ABSTRACT

The present invention relates to novel nutritional compositions and methods to mitigate inadequate nutrient intakes of intermittent fasting (IF) diets, in particular time restricted feeding (TRF). It also covers an Artificial Intelligence (AI)-based system for determination, quantification and mitigation of nutritional risks of said TRF diets in adults.

### FIELD OF THE INVENTION

The present invention relates to novel nutritional compositions and methods to mitigate inadequate nutrient intake of intermittent fasting (IF) diets, in particular the time-restricted feeding regimen (TRF). It also covers an Artificial Intelligence (AI)-based system for determination, quantification and mitigation of nutritional risks of said TRF diet in adults.

### BACKGROUND TO THE INVENTION

Intermittent fasting (IF) is a generic term to describe dietary patterns that include a period where little or no energy intake is consumed, alternating with a feeding-sometimes referred to as feasting-period. One of the most popular styles of IF is the time-restricted feeding (TRF) regimen where consumption of food is only allowed during a specified period of time per day, e.g., people on a 16:8 regimen fast for 16 h and eat for 8 h each day.

Generally used as a weight loss regimen, TRF has also been purported to have other health benefits including glycemic control and cardiovascular health, based on animal and human research. Efficacy of TRF on health parameters varies widely in the literature, most likely due to the variability in study subjects and study designs.

A person following a TRF diet, typically will not be aware of possible nutrient inadequacies stemming from the diet or the amounts of those inadequacies. These two points cannot be easily answered by existing clinical trials because the real-life eating patterns may differ from the dietary choices given in a clinical setting, under controlled conditions and constant supervision, therefore, the nutrients at risk in clinical settings can differ from real-life settings. In addition, most randomized controlled trials were performed in obese or overweight populations; and the study sizes and number of subjects in previous studies are generally small. Finally, nutritional needs are dependent on age, gender, height, weight and individual conditions (pregnancy, lactation, etc.).

Also, constant daily reporting and analysis of individual's diet is not practicable because daily recording is tedious, people forget to report individual items and eating occasions, they do not have access to nutritional databases, and they lack the expertise needed to identify nutrient inadequacies.

Accordingly, there is a need to provide solutions in the form of nutritional compositions and methods to prevent nutrients inadequacies in individual following a time-restricted feeding (TRF) regimen for a prolonged period of time.

### SUMMARY OF THE INVENTION

The present invention addresses the inadequate nutrient intakes in the state of the art by providing new nutritional recommendations and innovative methods for personalized nutrient, dietary and lifestyle recommendations for individual who are following or planning to follow TRF regimen (e.g., 16:8, 20:4, 12:12).

In one aspect, the present invention provides a computer implemented method or AI based system to identify and/or quantify individual nutritional inadequacies in individuals following or planning to follow a time-restricted feeding regimen, comprising:
(i) Identifying the individual's nutritional needs and/or dietary rules,
(ii) Comparing the individual's nutritional needs and/or dietary rules with the nutrients provided by a TRF diet, and
(iii) Performing analyses to identify and quantify the potential nutritional inadequacies.

In an embodiment, the individual's nutritional needs are based on one or more of the following personal characteristics: age, gender, height, weight, physical activity, lifestyle and medical condition.

In another embodiment the dietary rules are based on individual's specific dietary restrictions (e.g., gluten-free, lactose-free, etc.) or preferences (e.g., Mediterranean Diet, Flexitarian, Vegetarian (with and without dairy and eggs), Vegan, etc.).

In an embodiment the TRF diet is a simulated TRF diet.

In another aspect, the present invention addresses the specific condition of dietary inadequate intakes of a TRF regimen, by providing novel, consolidated dietary recommendations. More particularly, the present invention provides a computer implemented method or AI-based system to mitigate nutrient inadequacies in an individual following or planning to follow a TRF regimen, said method comprising:
i) Identifying nutritional inadequacies in the context of a defined TRF diet,
ii) Providing specific dietary recommendations and/or nutritional solutions to mitigate the identified nutritional inadequacies in said individual, and
iii) Optionally providing recommendations on lifestyle.

In an embodiment, the method to identify nutritional inadequacies is as described above so that, in another aspect, the present invention provides a method of mitigating nutrient inadequacies in an individual following or planning to follow a TRF regimen, said method comprising:
i) Identifying the individual's nutritional needs and/or dietary rules,
ii) Comparing the individual's nutritional needs and/or dietary rules with the nutrients provided by a TRF diet,
iii) Performing analyses to identify and quantify potential nutritional inadequacies,
iv) Providing specific dietary recommendations and/or nutritional solutions to mitigate the identified nutritional inadequacies in said individual, and
v) Optionally providing recommendations on lifestyle.

In an embodiment, the nutritional solutions include foods, beverages and/or dietary supplements thus recommended in the context of defined TRF dietary pattern, lifestyle and dietary rules.

An advantage of the present invention is to aid dietary decision-making before the user takes the decision to change its habitual diets and guide on needed dietary habit changes when following an TRF diet. It provides an estimation of prospective nutritional status by simulating the chosen diet and analyzing possible nutritional gaps in advance.

Another advantage of the present invention is to help individuals following or planning to follow an TRF diet to establish a personalized nutritional risk mitigation plan. In particular, it can provide awareness to diet followers on nutritional risks of inadequate intake when following such a diet via AI-based and nutrition science models.

A further advantage of the present invention is to prompt users to consume foods, beverages and/or dietary supplements to fulfill nutrient requirements specific to their TRF regimen.

It also provides an analysis to estimate the proportion of US adults following temporal eating patterns (i.e. skipping meals) that might indicate adherence to a TRF regime, and to identify their demographic characteristics. In addition, by looking at their nutrient intakes, one can infer whether restricted feeding is associated with diet quality (as measured by nutrient density), in addition to quantity.

### BRIEF DESCRIPTION OF FIGURES

FIG. 1. Calcium intake ranges comparing simulated baseline intakes with 16:8 TRF for males. The boxes show the 25^{th} percentile (lower bound) and 75^{th} percentile (upper bound) of intakes for each diet. The bar in the middle of each box is the Median intake. Median intakes at baseline exceed the RDA for calcium, but above the EAR. The 16:8 TRF regimen has median intakes lower than the median at baseline.

FIG. 2. Calcium intake ranges comparing simulated baseline intake with 16:8 TRF for females. The boxes show the 25^{th} percentile (lower bound) and 75^{th} percentile (upper bound) of intakes for each diet. The bar in the middle of each box is the Median intake. Median intakes at baseline exceed the RDA for calcium, but above the EAR. The 16:8 TRF regimen has median intakes lower than the median at baseline.

FIG. 3. Fiber intake ranges comparing simulated baseline intakes with 16:8 TRF for males. The boxes show the 25^{th} percentile (lower bound) and 75^{th} percentile (upper bound) of intakes for each diet. The bar in the middle of each box is the Median intake. Fiber intakes at baseline and for 16:8 TRF diet are below the AI.

FIG. 4. Fiber intake ranges comparing simulated baseline intakes with 16:8 TRF for females. The boxes show the 25^{th} percentile (lower bound) and 75^{th} percentile (upper bound) of intakes for each diet. The bar in the middle of each box is the Median intake. The Median intakes of fiber for females at baseline is above the AI. Median intakes for fiber 16:8 TRF are below the AI.

### DETAILED DESCRIPTION OF THE INVENTION

### Definitions

Prior to discussing the present invention in further details, the following terms and conventions will first be defined.

Examples of types of Time Restricted Feeding (TRF) include the following:
12:12—fasting for 12 hours and ad libitum eating for 12 hours,
16:8—fasting for 16 hours and ad libitum eating for 8 hours, and
20:4—fasting for 20 hours and ad libitum eating for 4 hours.
   Within the context of the present invention, “nutrients” are substances needed for health, growth, development and functioning of an organism, including:
Macronutrients (e.g., protein, carbohydrates, fats) and their components (e.g., amino acids, sugars, starches, fatty acids, etc.),
Micronutrients (e.g., vitamins, minerals),
Other food components (fiber, cholesterol, bioactive phytochemicals, alcohol, etc.),
Water contained in foods and beverages.

The term “composition” can mean a food, beverage, dietary supplement, complete nutrition or oral nutritional supplement (ONS) or medical food composition, or mixture thereof.

Within the context of the present invention, the terms “food,” “food product” and “food composition” mean a product or composition that is intended for ingestion by an individual such as a human and provides nutritional support to an organism, including those that provide energy, nutrients, and water. The compositions of the present disclosure, including the many embodiments described herein, can comprise, consist of, or consist essentially of one or more of the nutrients listed above, as well as any additional or optional ingredients or components safe for human consumption and otherwise useful in a diet.

Within the context of the present invention, the term “beverage”, “beverage product” and “beverage composition” mean a potable liquid product or composition for ingestion by an individual such as a human and provides water and may also include one or more nutrients and other ingredients safe for human consumption to the individual. The compositions of the present disclosure, including the many embodiments described herein, can comprise, consist of, or consist essentially of one or more of the nutrients listed above, as well as any additional or optional ingredients, components safe for human consumption and otherwise useful in a diet.

Within the context of the present invention, “dietary supplements” are products taken by mouth that contain one or more dietary ingredient, such as vitamins, minerals, amino acids, fatty acids, fibers and/or herbs and other botanical ingredients used to supplement the diet. Dietary supplements come in many forms and may be available as tablets, capsules, powders, liquids, and formulated into specific foods, such as “energy” bars.

As used herein, “complete nutrition” contains sufficient types and levels of macronutrients (protein, fats and carbohydrates), micronutrients, and other food components to be sufficient to be a sole source of nutrition for the subject to which the composition is administered. Individuals can receive 100% of their nutritional requirements from such complete nutritional compositions.

Within the context of the present invention, the term “Dietary Reference Intakes (DRIs)” indicates a set of reference values used to plan and assess the nutrient intakes of healthy people. The DRIs were established by the United States and Canadian governments, and published by the National Academies of Sciences, Engineering, and Medicine (NASEM; formerly called Institute of Medicine (IOM); https://www.nal.usda.gov/fnic/drinutrient-reports). Within the context of the present invention, the terms used to describe the DRIs (Institute of Medicine (US) Food and Nutrition Board. Dietary Reference Intakes: A Risk Assessment Model for Establishing Upper Intake Levels for Nutrients. Washington DC, USA: National Academies Press; 1998. What are Dietary Reference Intakes?) are as follows:
Recommended Dietary Allowance (RDA) indicates the average daily intake level of a nutrient considered sufficient to meet the requirements of 97.5% of healthy individuals. RDAs vary by gender, age, and whether a woman is pregnant or lactating. RDAs are calculated based on the Estimated Average Requirements (EAR) and is usually approximately 20% higher than the EAR.
Adequate Intake (AI) for a nutrient is the amount estimated to meet or exceed the amount needed to maintain adequate nutrition for most people in a specific age or gender group. An AI is set instead of an RDA when there is insufficient evidence to calculate an EAR.
Tolerable Upper Intake Level (UL) is the highest amount of daily nutrient intake considered to pose no risk of adverse health effects for most people. Consuming more than the UL increases the risk of adverse effects from over-consumption.
Estimated Average Requirement (EAR) for nutrients are calculated to meet the requirements of 50% of the people in a specific age and gender group.

The EAR is required to establish an RDA. If the standard deviation (SD) of the EAR is available and the requirement for the nutrient is symmetrically distributed, the RDA is set at two SDs above the EAR:

$$\\ RDA = EAR + 2 SD ( EAR )$$

If data about variability in requirements are insufficient to calculate a SD, a coefficient of variation (CV) for the EAR of 10 percent is assumed, unless available data indicate a greater variation in requirements. If 10 percent is assumed to be the CV, then twice that amount when added to the EAR is defined as equal to the RDA. The resulting equation for the RDA is:

$$\\ RDA = 1.2 ( EAR )$$

Different national and regional authorities have different dietary reference values. For example, The European Food Safety Authority (EFSA) refers to the collective set of information as Dietary Reference Values (DRV), with Population Reference Intake (PRI) instead of RDA, and Average Requirement instead of EAR. AI and UL are defined the same as in United States, but values may differ (EFSA Panel on Dietetic Products, Nutrition, and Allergies (NDA). Scientific Opinion on Principles for Deriving and Applying Dietary Reference Values. EFSA J. 2020; 8(3):1458. https://doi.org/10.2903/j.efsa.2010.1458). These standards and values are also used in the context of this invention.

Within the context of the present invention, the terms “nutrient inadequacy” or “dietary inadequacy” indicates that the total daily dietary intake of a nutrient in a certain individual is below the Estimated Average Requirements (EARs) for said individual and/or below well-established nutritional requirements.

Within the context of the present invention, the expression “prevent nutrient inadequacy” should be understood to include prevention of inadequacies of the nutrient or nutrients, as well as reduction of the risk of nutrient inadequacies in the individual following TRF diet.

Within the context of the present invention, numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 1 to 10 (including 1 and 10), from 2 to 8, from 3 to 7, from 1 to 9, from 3.6 to 4.6, from 3.5 to 9.9, and so forth. All references to singular characteristics or limitations of the present invention shall include the corresponding plural characteristic or limitation, and vice versa, unless otherwise specified or clearly implied to the contrary by the context in which the reference, is made.

Within the context of the present invention, the term “and/or” used in the context of the “X and/or Y” should be interpreted as “X”, or “Y”, or “X and Y”.

Unless defined otherwise, all technical and scientific terms used herein have the same meaning as commonly understood by one of ordinary skill in the art.

### Embodiments

The present invention provides a computer implemented method or AI-based system for providing nutritional recommendations to mitigate nutrient inadequacies in subjects following or planning to follow a Time-Restricted Feeding (e.g., 16:8, 20:4, 12:12).

In a first aspect, the present invention provides a computer implemented method or AI-based system to identify and/or quantify individual's nutritional inadequacies in an individual following or planning to follow a TRF regimen, comprising the steps of:
(i) Identifying the individual's nutritional needs and/or dietary rules,
(ii) Comparing the individual's nutritional needs and dietary rules with the nutrients provided by a TRF diet, and
(iii) Performing analyses to identify and quantify potential nutritional inadequacies.

In an embodiment, the Time-Restricted Feeding will focus, as an example, on 16:8 protocol, since it is the most popular protocol among consumers based on internal consumer insights data. In another embodiment, the TRF may also be 20:4 or 12:12 or any variations of the said protocols 16:8, 20:4, 12:12.

In another embodiment, the individual's nutritional needs are based on one or more of the following individual's characteristics: age, gender, height, weight, physical activity, lifestyle and medical condition. In a preferred embodiment, the nutritional needs are based at least on gender, age, weight height and physical activity.

In another embodiment the dietary rules are based on individual's specific dietary restrictions (e.g., gluten-free, lactose-free, etc.) or preferences (e.g., Mediterranean Diet, Flexitarian, Vegetarian (with and without dairy and eggs), Vegan, etc.).

General Analysis of Nutritional Inadequacies from National Health and Nutrition Examination Survey (NHANES) US Epidemiological Data Methodology

In a preferred embodiment, a statistical analysis was performed to identify potential nutrient inadequacies associated to TRF diet. Since NHANES survey does not collect information on fasting, the analysis was performed to mimic as close as possible voluntary time-restricted feeding dietary patterns. By making certain assumptions, the analysis can provide a reasonable estimate of the nutritional gaps and thus provide recommendations on how to close those gaps without requiring undue effort or input from the user.
1. Dietary intake data and demographics were used from several cycles of NHANES survey.
2. For each subject, different time intervals between consecutive meals were calculated.
3. Several inclusion and exclusion criteria were applied (e.g. >5000 kCal/day as exclusion criteria).
4. Food insecurity was also taken into account to exclude those individuals who were skipping a meal due to their socioeconomical status.
5. Perform analysis to identify and quantify potential nutritional inadequacies/gaps.

In another embodiment the TRF diet is a simulated TRF diet.

### General Diet Simulator Methodology

In a preferred embodiment, to simulate the TRF diet and identify potential nutrient inadequacies associated to it, we conduct the following steps:

1. Simulate N=20000 possible days of dietary intakes, 10000 each for males and females, where days of dietary intakes were defined as a set of foods and beverages and their amounts assigned for different eating occasions (e.g., breakfast, lunch, dinner and 1 snack) per day, where each simulation follows certain requirements:
Meet the person's energy needs based on age, gender, height, weight, physical activity level,
Follow TRF rules, defined by diet protocol type (e.g., 16:8, 20:4, 12:12), and
Provide realistic amounts of foods and beverages and their combinations to generate daily nutrient totals.

2. Compare the person's individual nutritional needs (depending on age, gender, height, weight, physical activity, etc.) with the nutrients provided by the simulated TRF diet.

3. Perform analysis to identify and quantify potential nutritional inadequacies/gaps.

Ultimately, said method is used to recommend foods, beverages and dietary supplements that best close the identified nutritional inadequacies/gaps in the context of defined Time-Restricted Feeding dietary patterns.

By making certain assumptions, the system or method can provide a reasonable estimate of the nutritional gaps and thus provide recommendations on how to close those gaps without requiring undue effort or input from the user.

It is important to note that specific nutritional inadequacies/gaps will depend on dietary habits and cultural characteristics and will likely differ in different populations around the world. The system or method described here will be the same, but the nutritional inadequacies identified may differ. The example provided hereafter, relies on food group level diet recommendations as specified by the USDA Healthy Eating Patterns which provide target amounts of food groups to be consumed on a daily and weekly basis. The simulation tool can easily be adapted to different food-based dietary guidelines if applied in other geographies.

In another aspect, the present invention aims to provide novel, consolidated dietary recommendations to address the specific condition of dietary inadequate intake of an intermittent fasting diet, which combine:
Specific dietary intake amounts by gender, age, and intermittent fasting protocol followed or to be followed,
Specific dietary recommendations to be consumed daily by gender and intermittent fasting protocol followed or to be followed, and
Specific recommendations on lifestyle components (e.g., physical activity).

More particularly, the present invention provides a computer implemented method or AI-based system to mitigate nutrient inadequacies in an individual following or planning to follow an TRF regimen, said method comprising the steps of:
i) Identifying nutritional inadequacies in the context of a defined TRF dietary pattern, lifestyle and/or dietary rules,
ii) Providing specific dietary recommendations and/or nutritional solutions to mitigate the identified nutritional inadequacies in said individual, and
iii) Optionally providing recommendations on lifestyle.

In an embodiment, the method to identify and/or quantify the nutritional gaps is as described above. Additional criteria could be specified such as foods, food groups, and/or specific nutrients which should be excluded, minimized or maximized.

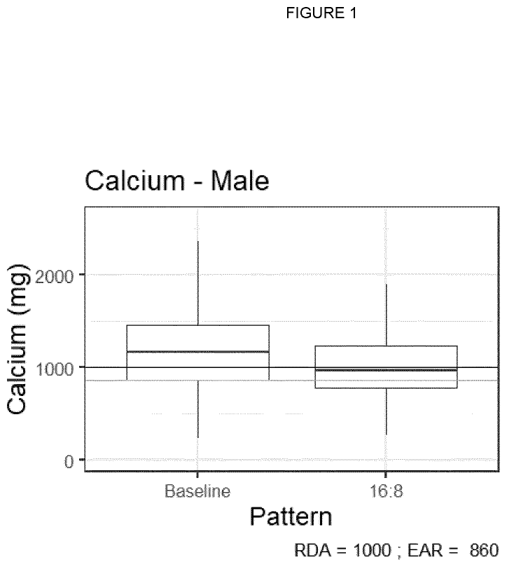

In a preferred embodiment, in order to construct dietary recommendations (as described in more details in the example section), we consider the upper and lower bounds of the intake recommendations to identify nutrient targets, without exceeding the UL (see FIGS. 1-4 for examples for calcium and folate). The “Amount of Inadequacy” is used to generate the dietary recommendations. First, we take the difference between the RDA, or the AI if no RDA is available (or any relevant guideline), and the 2.5% intake percentile as the upper and the difference with the 97.5% intake percentile as the lower. It is understood that any other percentile can be used. All recommendations are preferably based on an energy level for the diet of 2000 kcal. Alternatively, the recommendation may be based on a different energy level if desired. The upper end of the recommendation is capped to ensure that the recommendation does not exceed the UL.

In a further embodiment, a computerized Diet Simulator may be used. By making certain assumptions, the Diet Simulator can provide a reasonable estimate of the nutritional inadequacies/gaps and thus provide recommendations on how to close those gaps from nutrients, foods, beverages, and/or dietary supplements, and their combinations, without requiring undue effort or input from the user.

In an embodiment, the dietary recommendations comprise recommendations related to one or more of the following:
Nutrients, and/or
Foods and beverages, and/or
Dietary supplements, and/or
Combinations of foods and beverages as menu recommendations and/or recipe recommendations.

In an embodiment, the recommendations for nutrients, foods, beverages, and dietary supplements are selected to best close the identified nutritional inadequacies/gaps in the context of the defined TRF regimen. Said recommendations are based on the individual's characteristics, diet rules and TRF dietary pattern.

The system according to the present invention is a Diet Simulator, comprised of the following components:
A food composition database,
A recipe database,
A database of meals,
A nutrient scoring module, and
An optimization module.

The compositions disclosed herein are intended to be consumed orally. As such, non-limiting examples of the form of the composition include natural foods, processed foods (including but not limited to milling, grinding, baking, drying, fermenting, canning, freezing, pasteurizing, extruding, cooking, and other processing methods to make raw food ingredients palatable and ready-to-eat), creamers, beverages such as coffee based beverages, natural juices, concentrates and extracts.

In several embodiments, the present invention may include menu suggestions or recipes that incorporate foods, beverages and/or dietary supplements to alleviate the nutritional inadequacies or excesses caused by the TRF regimen. Depending on the TRF regimen, these foods, beverages, and/or dietary supplements as well as the menus and recipes that use them can be recommended for consumption on a daily basis, or on eating days for the duration of the individual's adherence to the TRF regimen.

In several embodiments, the present invention may also package foods, beverages and/or dietary supplements into an TRF “kit” of parts comprising meal recommendations, recipes, or menus for individuals on an TRF diet, wherein said kit includes a composition according to the invention.

### EXAMPLES

### Example 1: Analysis of Nutritional Inadequacies from National Health and Nutrition Examination Survey (NHANES) US Epidemiological Data

The National Health and Nutrition Examination Survey (NHANES) is a program of studies designed to assess the health and nutritional status of adults and children in the United States. The survey is unique in that it combines interviews and physical examinations. The NHANES interview includes demographic, socioeconomic, dietary, and health-related questions. The examination component consists of medical, dental, and physiological measurements, as well as laboratory tests administered by highly trained medical personnel. The sample for the survey is selected to represent the U.S. population of all ages. To produce reliable statistics, NHANES over-samples persons 60 and older, African Americans, and Hispanics. The cycles of data collection started in 1999 and it continues up to the date.

For this example, the NHANES cycles 2013-2014, 2015-2016, 2017-2018 were used, more specifically, the files “Dietary Interview-Individual Foods, First Day”, and “Demographic Variables and Sample Weights”. Datasets were downloaded from https://wwwn.cdc.gov/nchs/nhanes/default.aspx.

The dietary data consists of 1 or 2 24 h recalls, performed with the Automated Multiple-Pass Method. We analyzed only the first 24 h recall of each subject. From the dietary file, we extracted the list of foods reported by each individual, the time in HH:MM:SS, the name of the meal and the KCAL. This was merged with the demographics file to retrieve the age. A filter≥18 was applied to the age. Eating/drinking occasions providing no calories (e.g. water, black coffee without sugar) were not considered, so that a single glass of water was not considered as an ‘eating occasion.’

### Calculation

For each subject, the time intervals between consecutive meals were calculated. For example, if meals were reported at 7:00, 12:00, 16:00, 20:00, then the time intervals are: 7 h, 5 h, 4 h, 4 h, 4 h.

### Descriptive Statistics

First and last time of eating. The number of subjects having reported food intake before 5 AM was n=1206.
Time-span of eating. For each subject, we report the time interval ‘Last time of eating—First time of eating.’
Counts of fasting intervals. For the rest of the analysis, we exclude subjects with first eating time before 5 AM. The interval between consecutive meals ranges from a few minutes to 23.5 hours, with a median of 3.5 hours.
The fasting intervals chosen for the analysis were as follow:
   <12 hours fasting (n=64940)
   12 hours to 16 hours fasting (n=4717)
   >16 hours fasting (n=761). People in this group have reported eating times with a ‘gap’ of at least 16 h, for example no food consumption between 6 AM and 10 PM.
A cut-off of 5000 kcal was applied afterwards. All energy intakes above the cut-off were removed from the analysis.
The analysis was repeated after excluding respondents who declared to have restricted their food intake because of insufficient money. It should be noted that the rate of non-response to the Food Security Questionnaire is very high (>50%). The number of people within the different fasting intervals, and after applying the Food Security Questionnaire filter were as follows:
   <12 hours fasting (n=14088),
   12 hours to 16 hours fasting (n=1439), and
   >16 hours fasting (n=129).

The following question from the Food Security Questionnaire was used to identified people restricting their food intake due to socioeconomical status:
“In the last 12 months, since last month, did {you/you or other adults in your household} ever cut the size of your meals or skip meals because there wasn't enough money for food?”
   1: yes
   2: No
   9: Don't know

In NHANES, there are no questions related to intentional fasting, so it is not possible to make a direct connection between these intervals and a TRF regime. However, we assumed that the nutrient needs of people restricting eating to an 8-h interval would be similar to those intentionally following a 16:8 TRF diet.

### Time-Restricted Feeding Intervals Analysis

Macronutrient intakes were calculated for the three chosen fasting intervals, <12 h, 12 h to 16 h, and >16 h (Table 1). The repartition of energy among fats, proteins and carbohydrates is similar between the three groups; means and medians fall within the acceptable macronutrient distribution ranges: Fats (20-35% of energy), Protein (10-35% of energy) and Carbohydrate (45-65% of energy), as defined by the US National Academy of Sciences (https://ods.od.nih.gov/Healthlnformation/Dietary_Reference_Intakes.aspx).

TABLE 1
Macronutrient intakes as a percentage of energy, by fasting interval.

| TRF | Fat intake | Protein intake | Carbohydrate intake |
|---|---|---|---|
| period | % Daily Energy | % Daily Energy | % Daily Energy |
| <12 h | Mean (SD): 33.6 (9.29) | Mean (SD): 15.8 (5.59) | Mean (SD): 49.5 (11.5) |
| (N = 1858) | Median: 34.0 | Median: 14.9 | Median: 49.6 |
| | Min, Max: 2.76, 66.2 | Min, Max: 1.81, 50.3 | Min, Max: 12.2, 91.8 |
| 12 h-16 h | Mean (SD): 33.6 (9.95) | Mean (SD): 15.6 (6.04) | Mean (SD): 48.3 (11.4) |
| (N = 250) | Median: 34.8 | Median: 14.8 | Median: 47.4 |
| | Min, Max: 1.51, 58.8 | Min, Max: 0.401, 40.5 | Min, Max: 19.7, 89.1 |
| >16 h | Mean (SD): 34.3 (10.1) | Mean (SD): 16.5 (7.59) | Mean (SD): 47.9 (12.9) |
| (N = 48) | Median: 35.8 | Median: 14.8 | Median: 48.2 |
| | Min, Max: 6.10, 55.9 | Min, Max: 7.46, 46.9 | Min, Max: 12.1, 79.8 |
| Overall | Mean (SD): 33.6 (9.39) | Mean (SD): 15.8 (5.69) | Mean (SD): 49.3 (11.5) |
| (N = 2156) | Median: 34.0 | Median: 14.9 | Median: 49.4 |
| | Min, Max: 1.51, 66.2 | Min, Max: 0.401, 50.3 | Min, Max: 12.1, 91.8 |

Micronutrient intakes were calculated for the three chosen fasting intervals, <12 h, 12 h to 16 h, and >16 h (Table 2). All the nutrients considered have statistically significant distributions between the groups “<12 h” and “>16 h” (Wilcoxon test, p<0.05), even after adjusting for energy cofounders (adjusted by 1000 kCal).

TABLE 2
Distributions of micronutrient intakes per 1000 kcal, by fasting interval

| Nutrient | Group | Minimum | Quartile1 | Mean | Median | Quartile3 | Max |
|---|---|---|---|---|---|---|---|
| Calcium | <12 h | 41.2 | 249.5 | 405.3 | 371.6 | 521.5 | 1675.4 |
| | 12 h-16 h | 37.5 | 196.1 | 345.0 | 315.7 | 450.8 | 1042.5 |
| | >16 h | 49.6 | 139.3 | 338.2 | 281.8 | 473.3 | 1140.6 |
| Fiber | <12 h | 0.1 | 5.0 | 8.4 | 7.3 | 10.6 | 66.9 |
| | 12 h-16 h | 0.7 | 4.5 | 7.4 | 6.2 | 8.9 | 39.0 |
| | >16 h | 1.7 | 4.2 | 7.2 | 5.7 | 8.0 | 22.7 |
| Folate | <12 h | 12.6 | 125.9 | 194.9 | 169.2 | 235.8 | 3128.3 |
| | 12 h-16 h | 24.3 | 88.2 | 158.2 | 132.5 | 199.6 | 415.6 |
| | >16 h | 0.0 | 107.1 | 164.9 | 147.5 | 205.9 | 834.5 |
| Iron | <12 h | 1.1 | 4.9 | 7.0 | 6.2 | 7.9 | 46.6 |
| | 12 h-16 h | 1.3 | 4.4 | 6.2 | 5.8 | 7.3 | 19.8 |
| | >16 h | 2.1 | 4.2 | 6.0 | 5.9 | 7.1 | 12.5 |
| Magnesium | <12 h | 13.4 | 98.3 | 133.3 | 124.0 | 156.4 | 995.0 |
| | 12 h-16 h | 30.4 | 87.2 | 119.5 | 108.9 | 145.0 | 347.4 |
| | >16 h | 53.9 | 82.6 | 118.4 | 97.4 | 127.8 | 359.8 |
| Niacin | <12 h | 0.9 | 8.7 | 12.4 | 11.2 | 14.5 | 59.8 |
| | 12 h-16 h | 0.5 | 8.8 | 12.8 | 11.4 | 15.3 | 89.3 |
| | >16 h | 4.2 | 8.6 | 13.6 | 11.8 | 16.1 | 57.6 |
| Potassium | <12 h | 144.7 | 938.6 | 1254.1 | 1185.6 | 1484.9 | 4269.9 |
| | 12 h-16 h | 297.2 | 832.9 | 1130.1 | 1065.0 | 1325.1 | 2612.6 |
| | >16 h | 389.7 | 801.7 | 1176.0 | 942.7 | 1352.4 | 2997.3 |
| Selenium | <12 h | 7.3 | 41.6 | 56.6 | 52.7 | 67.1 | 300.9 |
| | 12 h-16 h | 0.8 | 40.1 | 54.9 | 52.7 | 67.0 | 158.2 |
| | >16 h | 4.6 | 36.9 | 58.3 | 52.0 | 69.6 | 226.7 |
| Sodium | <12 h | 315.6 | 1309.5 | 1676.7 | 1599.5 | 1961.7 | 13041.6 |
| | 12 h-16 h | 82.6 | 1331.1 | 1688.0 | 1596.5 | 1940.8 | 4847.5 |
| | >16 h | 996.0 | 1361.5 | 1728.4 | 1649.7 | 2031.5 | 3339.5 |
| Vitamin A | <12 h | 0.0 | 124.8 | 284.6 | 218.5 | 345.5 | 4039.7 |
| | 12 h-16 h | 0.0 | 87.0 | 240.3 | 162.1 | 282.9 | 2931.9 |
| | >16 h | 6.2 | 73.0 | 228.9 | 148.7 | 252.5 | 1537.7 |
| Thiamin | <12 h | 0.1 | 0.6 | 0.8 | 0.7 | 0.9 | 5.4 |
| | 12 h-16 h | 0.0 | 0.5 | 0.7 | 0.6 | 0.8 | 3.0 |
| | >16 h | 0.2 | 0.5 | 0.7 | 0.6 | 0.8 | 1.4 |
| Vitamin B12 | <12 h | 0.0 | 1.1 | 2.3 | 1.7 | 2.8 | 45.8 |
| | 12 h-16 h | 0.0 | 0.9 | 2.1 | 1.5 | 2.7 | 24.6 |
| | >16 h | 0.0 | 0.7 | 1.8 | 1.4 | 1.9 | 12.7 |
| Riboflavin | <12 h | 0.2 | 0.7 | 1.0 | 0.9 | 1.1 | 7.6 |
| | 12 h-16 h | 0.1 | 0.5 | 0.9 | 0.7 | 1.0 | 13.1 |
| | >16 h | 0.1 | 0.6 | 0.8 | 0.7 | 0.8 | 4.6 |
| Vitamin B6 | <12 h | 0.1 | 0.6 | 1.0 | 0.9 | 1.2 | 27.4 |
| | 12 h-16 h | 0.0 | 0.6 | 1.0 | 0.8 | 1.2 | 8.8 |
| | >16 h | 0.3 | 0.5 | 1.0 | 0.8 | 1.3 | 5.5 |
| Vitamin C | <12 h | 0.0 | 10.1 | 41.1 | 24.1 | 55.4 | 621.3 |
| | 12 h-16 h | 0.0 | 6.3 | 39.0 | 17.6 | 49.2 | 323.0 |
| | >16 h | 0.2 | 4.6 | 39.4 | 12.9 | 42.2 | 369.9 |
| Vitamin D | <12 h | 0.0 | 0.6 | 2.3 | 1.7 | 3.0 | 31.9 |
| | 12 h-16 h | 0.0 | 0.3 | 1.7 | 0.8 | 2.1 | 26.9 |
| | >16 h | 0.0 | 0.2 | 1.6 | 0.6 | 1.3 | 21.3 |
| Vitamin E | <12 h | 0.1 | 2.5 | 3.9 | 3.5 | 4.6 | 37.1 |
| | >16 h | 1.2 | 2.5 | 3.9 | 3.6 | 4.2 | 12.5 |
| | 12 h-16 h | 0.1 | 2.5 | 3.7 | 3.4 | 4.5 | 16.2 |
| Zinc | <12 h | 0.8 | 3.7 | 5.2 | 4.7 | 6.0 | 70.0 |
| | 12 h-16 h | 0.7 | 3.4 | 5.0 | 4.5 | 5.7 | 15.2 |
| | >16 h | 1.3 | 3.1 | 4.4 | 4.0 | 5.1 | 14.2 |

### Risk of Inadequate Intakes of Time Restricted Feeding, by Fasting Intervals

Table 3 shows the risk for inadequacies (percentage below EAR, or AI when EAR was not available) identified by TRF fasting intervals.

| | Percent Below Recommendation* | | |
|---|---|---|---|
| | <12 h fasting | 12 h to 16 h fasting | >16 h fasting |
| Nutrient | N = 1858 | N = 250 | N = 48 |
| Calcium | 45.4 | 64.4 | 66.7 |
| Fiber | 90.2 | 95.2 | 97.9 |
| Folate | 47.3 | 61.2 | 81.2 |
| Iron | 14.7 | 26.8 | 45.8 |
| Magnesium | 65.6 | 79.2 | 89.6 |
| Potassium | 95.7 | 98.0 | 97.9 |
| Vitamin A | 63.0 | 77.6 | 81.2 |
| Vitamin B12 | 25.7 | 38.0 | 54.2 |
| Vitamin C | 60.8 | 68.4 | 79.2 |
| Vitamin D | 90.5 | 94.4 | 97.9 |
| Vitamin E | 83.8 | 87.6 | 95.8 |
| Zinc | 38.4 | 50.0 | 72.9 |

*Percent below the Estimated Average Requirement, except for potassium and fiber, where the percent below the Adequate Intake is shown.

### Example 2: Risk of Inadequate Nutrient Intakes of Intermittent Fasting Protocols with Simulated Diets

In order to estimate the potential risk of nutrients inadequacies for TRF diet, without the use of randomized controlled trials (RCT) or traditional dietary assessment methods (e.g., 24-hours food recall, food frequency questionnaire, food records), we simulated approximately 20,000 days of food intake in silico for a variety of individuals adhering to an TRF diet with a digital tool. For this example, 16:8 protocol was used as an illustrative example of a TRF diet.

The digital simulation tool simulates multiple days of food intake by finding the optimal combination of available meals to maximize the nutritional balance of the meals by conforming as best as possible to the USDA Healthy Eating Plan guidelines. In order to mimic real-world food intake as closely as possible, we used actual meals consumed by people as reported in the National Health and Nutrition Examination Survey (NHANES), a survey performed by the US Center for Disease Control and Prevention (CDC). Datasets were downloaded from https://wwwn.cdc.gov/nchs/nhanes/default.aspx. The NHANES cycles 2013-2014, 2015-2016, 2017-2018 were used, more specifically, the files “Dietary Interview-Individual Foods, First Day,” and “Demographic Variables and Sample Weights.” This allows for the simulated diets to consist of meals actually consumed by the US population.

It is important to note that specific nutritional inadequacies will depend on dietary habits and cultural characteristics and will likely differ in different populations around the world. The system or method described here will be the same, but the nutritional inadequacies identified may differ.

Within the simulation tool, the user specifies the characteristics for a set of individuals based on sex, age, height, weight and physical activity level in order to calculate the estimated energy requirement (EER) per day. The simulation tool then uses integer programming techniques to create in silico menu plans which optimize the nutritional content of the overall diet. In this Example, the simulation tools rely on food group level diet recommendations as specified by the USDA Healthy Eating Patterns which provide target amounts of food groups to be consumed on a daily and weekly basis. (The simulation tool can be adapted to different food-based dietary guidelines if applied in other geographies.)

Additional nutrient level constraints are set as upper limits on nutrients such as sodium, added sugars, and saturated fats. The constraints are adjusted based on the individual's EER to obtain desired ranges of food groups and nutrients to be consumed per day and per week. An objective function is created as a scalar weighted linear combination of the differences between the actual amount of each individual food group and nutrient and the optimal range. The diet is created by selecting the combination of meals which meet the energy requirements while attempting to keep the consumption of food groups and nutrients within the optimal range.

### Baseline Diet Simulation

To simulate the dietary intakes of TRF regimen (e.g., 16:8), we simulated approximately 20,000 days of “baseline” diet by maximizing the Healthy Eating Patterns as described above with no other rules applied. This “baseline” diet is considered to be a healthy and balanced diet (Table 4). In this Example, the baselines were simulated for 1,400 individuals of each gender, ranging in age from 18 to 70, of varying heights and weights and sedentary physical activity level, for 7 days each. While the use of meals extracted from NHANES forces the simulated diets to use meals reportedly consumed by actual people, the optimization of the balance of food groups produces diets with a bias towards healthier diets than what people are likely to actually eat. As such we considered these to be “ideal” diets rather than realistic diets, which will tend to have higher nutrient intakes than occur in the real world.

TABLE 4
Percent of individuals with inadequate intakes
at baseline (from diet simulation models).

| | Males | | | Females | | |
|---|---|---|---|---|---|---|
| | Mean Intake | | Percent | Mean Intake | | Percent |
| Nutrient | Baseline | | Inadequate | Baseline | | Inadequate |
| Total fat | 47.1 | g | 0 | 41.7 | g | 0 |
| Fiber | 28.9 | g | 77 | 27.4 | g | 17 |
| Calcium | 1240.1 | mg | 9 | 1090.7 | mg | 41 |
| Copper | 1.7 | μg | 0 | 1.5 | μg | 0 |
| Food folate | 387.4 | μg | 12 | 376.0 | μg | 30 |
| Iron | 14.3 | mg | 0 | 12.2 | mg | 3 |
| Magnesium | 402.3 | mg | 2 | 370.8 | mg | 4 |
| Niacin | 20.9 | mg | 0 | 17.4 | mg | 7 |
| Potassium | 5121.9 | mg | 0 | 4620.0 | mg | 0 |
| Riboflavin | 2.3 | mg | 0 | 2.0 | mg | 0 |
| Selenium | 93.2 | μg | 0 | 77.4 | μg | 9 |
| Sodium | 2256.2 | mg | 1 | 1931.4 | mg | 24 |
| Vitamin A | 1076.1 | μg | 9 | 875.3 | μg | 23 |
| Vitamin B12 | 4.2 | μg | 2 | 3.3 | μg | 17 |
| Vitamin B6 | 3.0 | mg | 0 | 2.6 | mg | 1 |
| Vitamin C | 496.4 | mg | 0 | 479.5 | mg | 0 |
| Vitamin D | 7.5 | μg | 59 | 5.6 | μg | 86 |
| Vitamin E | 8.2 | mg | 94 | 8.0 | mg | 93 |
| Vitamin K | 113.7 | μg | 80 | 122.2 | μg | 52 |
| Zinc | 10.6 | mg | 18 | 9.0 | mg | 25 |

It is noted that some nutrients are low at baseline, meaning they are not at risk particularly because of IF diets, but in general in US diet/baseline.

### 16:8 TRF Protocol Diet Simulation

The 16:8 TRF protocol was created from the baseline diet by removing meals as per the rules of the protocol: 16 h fasting and 8 hours eating. In this case, the simulation was done by skipping breakfast meal occasion.

By comparing the baseline inadequacies to the different TRF protocols, we can identify how nutrient intakes change when following this type of TRF protocol (Table 5). This method of simulation, where a baseline diet is modified to comply with the rules of the IF protocol, forces the results to be comparable, allowing us to identify the relative risks of nutrient inadequacy for the TRF diet while controlling for randomness inherent in the simulation process and for nutrient inadequacies which may result from the simulation process rather than being inherent in the TRF regimen.

TABLE 5
Percent of individuals with inadequate intakes when rules for
16:8 TRF regimen were applied (from diet simulation models).

| | Percent of | Percent of | Percent of |
|---|---|---|---|
| | Inadequacy, | Inadequacy, | Inadequacy, both |
| Nutrient | Males | Females | Males and Females |
| Calcium | 29.0% | 55.0% | 42.0% |
| Copper | 0.0% | 3.0% | 1.0% |
| Fiber | 95.0% | 69.0% | 82.0% |
| Food Folate | 33.0% | 52.0% | 42.0% |
| Iron | 0.0% | 13.0% | 7.0% |
| Magnesium | 41.0% | 28.0% | 35.0% |
| Niacin | 1.0% | 14.0% | 8.0% |
| Potassium | 1.0% | 1.0% | 1.0% |
| Riboflavin | 4.0% | 8.0% | 6.0% |
| Selenium | 0.0% | 15.0% | 8.0% |
| Sodium | 2.0% | 34.0% | 18.0% |
| Vitamin A | 18.0% | 30.0% | 24.0% |
| Vitamin B12 | 17.0% | 43.0% | 30.0% |
| Vitamin B6 | 0.0% | 9.0% | 5.0% |
| Vitamin C | 0.0% | 0.0% | 0.0% |
| Vitamin D | 85.0% | 93.0% | 89.0% |
| Vitamin E | 99.0% | 98.0% | 99.0% |
| Vitamin K | 84.0% | 58.0% | 72.0% |
| Zinc | 43.0% | 42.0% | 43.0% |

The diet for each simulated individual is analyzed by comparing the mean intakes of each nutrient to the DRI for that individual, based on their age and gender. As nutrient intakes tend to not be normally distributed, bootstrap confidence intervals are created for the mean which are compared to the appropriate DRI. In order to pool results for individuals with varying DRIs, the result for each individual is calculated as a ratio of their mean intake to their DRI. The overall gaps between simulated intake and DRI are created by taking the mean of these ratios. The risks of inadequacies are created by taking the percentage of simulated individuals with inadequate intake for each nutrient.

The results of the analyses and the resulting recommendations for each group are below. These tables show the complete results of the analysis.

### Risk of Inadequate Intakes of 16:8 TRF Reqimen

Tables 6-7 show the risk for inadequacies identified for males and females for 16:8 TRF regimen. Most nutrients have an EAR, but when there were not sufficient data to assign an EAR, an AI was assigned instead (Institute of Medicine, US). In the Tables below, “Mean Inadequacy” is the amount below EAR or AI. The “Percent Inadequate” indicates the percent of individuals below the EAR or AI. If a large portion of the modelled diets produce inadequacies, then those nutrients are considered to be “At Risk” for inadequacies. Note that the interpretation is somewhat different for the AI; with a percent above an AI, we can generally assume adequacy, but a percent below does not necessarily imply inadequacy. A negative Mean Inadequacy indicates that the intakes tend to be adequate.

TABLE 6
Nutritional Analysis of 16:8 TRF for Males.

| | | | EAR/AI | Mean | Percent | Assess- |
|---|---|---|---|---|---|---|
| Nutrient | Type | Units | Value | Inadequacy | Inadequate | ment |
| Fiber | AI | g | 34.8 | 10.9 | 94.8 | |
| Calcium | EAR | mg | 860 | −204.6 | 29.4 | |
| Copper | EAR | mcg | 0.7 | −0.8 | 00.0 | |
| Food Folate | EAR | mcg | 320 | −16.4 | 33.1 | At Risk |
| Iron | EAR | mg | 6 | −4.9 | 00.1 | |
| Magnesium | EAR | mg | 346 | −10.1 | 41.1 | At Risk |
| Niacin | EAR | mg | 12 | −7.1 | 1.4 | |
| Potassium | AI | mg | 3000 | −1665.7 | 0.6 | |
| Protein | EAR | g | 56 | −20.7 | 8.2 | |
| Riboflavin | EAR | mg | 1.1 | −0.8 | 3.7 | |
| Selenium | EAR | mcg | 45 | −50.6 | 0.4 | |
| Sodium | AI | mg | 1500 | −765.3 | 2.3 | |
| Vitamin A | EAR | mcg | 625 | −451.0 | 17.6 | |
| Vitamin B12 | EAR | mcg | 2.0 | −1.3 | 17.3 | |
| Vitamin B6 | EAR | mg | 1.22 | −1.4 | 0.0 | |
| Vitamin C | EAR | mg | 75 | −364.1 | 0.0 | |
| Vitamin D | EAR | mcg | 10 | 3.2 | 85.4 | |
| Vitamin E | EAR | mg | 12 | 5.1 | 98.7 | |
| Vitamin K | AI | mcg | 120 | 26.8 | 84.5 | |
| Zinc | EAR | mg | 9.40 | −0.3 | 43.4 | At Risk |

TABLE 7
Nutritional Analysis of 16:8 TRF for Females.

| | | | EAR/AI | Mean | Percent | Assess- |
|---|---|---|---|---|---|---|
| Nutrient | Type | Units | Value | Inadequacy | Inadequate | ment |
| Fat total | EAR | g | 18.4 | −18.4 | 0.4 | |
| Fiber | AI | g | 23.4 | 2.1 | 68.9 | |
| Calcium | EAR | mg | 880 | 25.1 | 55.1 | At Risk |
| Copper | EAR | mcg | 0.7 | −0.6 | 2.9 | |
| Food Folate | EAR | mcg | 320 | 7.5 | 51.6 | At Risk |
| Iron | EAR | mg | 6.86 | −2.4 | 13.1 | |
| Magnesium | EAR | mg | 263 | −36.6 | 28.4 | |
| Niacin | EAR | mg | 11 | −3.2 | 14.1 | |
| Potassium | AI | mg | 2300 | −1414.4 | 1.1 | |
| Protein | EAR | g | 46 | −9.6 | 23.4 | |
| Riboflavin | EAR | mg | 0.9 | −0.6 | 8.4 | |
| Selenium | EAR | mcg | 45 | −23.2 | 15.3 | |
| Sodium | AI | mg | 1500 | −252.0 | 34.4 | |
| Vitamin A | EAR | mcg | 500 | −254.7 | 29.5 | |
| Vitamin B12 | EAR | mcg | 2.0 | −0.4 | 43.3 | At Risk |
| Vitamin B6 | EAR | mg | 1.18 | −0.8 | 9.1 | |
| Vitamin C | EAR | mg | 60 | −316.2 | 0.0 | |
| Vitamin D | EAR | mcg | 10 | 5.7 | 92.8 | |
| Vitamin E | EAR | mg | 12 | 5.3 | 98.4 | |
| Vitamin K | AI | mcg | 90 | −25.0 | 58.5 | |
| Zinc | EAR | mg | 6.8 | −0.5 | 41.8 | At Risk |

### Analysis: Distributions of Intakes

Tables 8-9 show the distributions of intakes for the nutrients of interest. The nutrients of interest are selected as those which have a risk of inadequacy large enough to be notable, and where the percentage of simulated diets which have inadequate intakes differs significantly from the baseline.

TABLE 8
Distribution of Intakes for Nutrients of Risk for 16:8 TRF regimen, Males.

| Nutrient | Units | EAR/AI | RDA | Minimum | 2.5% | 50% | 97.5% | Maximum |
|---|---|---|---|---|---|---|---|---|
| Fiber | g | 34.8 | NA | 8.1 | 14.5 | 22.7 | 37.3 | 51.5 |
| Calcium | mg | 860 | 1000 | 269.4 | 358.1 | 968.2 | 2162.6 | 2667.4 |
| Food Folate | mcg | 320 | 400 | 33.7 | 134.7 | 340.3 | 503.8 | 573.5 |
| Magnesium | mg | 346 | 416 | 178.9 | 260.4 | 354.9 | 439.5 | 490.8 |
| Vitamin D | mcg | 10 | 15 | 0.0 | 0.5 | 6.1 | 19.3 | 75.7 |
| Vitamin E | mg | 12 | 15 | 3.1 | 4.4 | 6.4 | 12.2 | 19.3 |
| Vitamin K | mcg | 120 | NA | 7.3 | 26.8 | 74.3 | 290.4 | 1781.3 |
| Zinc | mg | 9.4 | 11 | 3.3 | 4.6 | 9.7 | 16.2 | 31.8 |

TABLE 9
Distribution of Intakes for Nutrients of Risk for 16:8 TRF regimen, Females.

| Nutrient | Units | EAR/AI | RDA | Minimum | 2.5% | 50% | 97.5% | Maximum |
|---|---|---|---|---|---|---|---|---|
| Calcium | mg | 880 | 1080 | 154.5 | 306.3 | 802.2 | 2125.7 | 2667.4 |
| Fiber | g | 23.4 | NA | 6.0 | 11.2 | 21.6 | 31.5 | 42.8 |
| Food Folate | mcg | 320 | 400 | 50.4 | 118.0 | 318.7 | 499.0 | 570.8 |
| Magnesium | mg | 263 | 318 | 127.8 | 173.3 | 303.8 | 403.8 | 514.5 |
| Potassium | mg | 2300 | NA | 1530.4 | 2237.0 | 3517.2 | 5669.2 | 6086.3 |
| Vitamin A | mcg | 500 | 700 | 1.8 | 119.2 | 702.7 | 1436.9 | 1776.7 |
| Vitamin B12 | mcg | 2.0 | 2.4 | 0.0 | 0.4 | 2.0 | 6.1 | 8.0 |
| Vitamin D | mcg | 10 | 15 | 0.0 | 0.1 | 3.7 | 14.7 | 20.5 |
| Vitamin K | mcg | 90 | NA | 5.2 | 19.0 | 80.1 | 331.4 | 582.1 |
| Zinc | mg | 6.8 | 8 | 2.1 | 3.5 | 7.4 | 11.3 | 17.0 |

### Analysis: Amount of Inadequacy

Tables 10-11 show the amount below the EAR or AI for each percentile (2.5%, 50% and 97.5%) for the nutrients of interest. The nutrients of interest are selected as those which have a risk of inadequacy large enough to be notable, and where the percentage of simulated diets which have inadequate intakes differs significantly from the baseline. If the values are negative it means that the intake tends to be above the DRI.

TABLE 10
Amount of inadequacy of each “At Risk”
nutrient for 16:8 TRF for Males.

| Nutrient | Units | EAR/AI | 2.5% Gap | 50% Gap | 97.5% Gap |
|---|---|---|---|---|---|
| Calcium | mg | 860 | 501.9 | −108.2 | −1302.6 |
| Fiber | g | 34.8 | 20.3 | 12.1 | −2.5 |
| Food Folate | mcg | 320 | 185.3 | −20.3 | −183.8 |
| Magnesium | mg | 346 | 85.6 | −8.9 | −93.5 |
| Vitamin D | mcg | 10 | 9.5 | 3.9 | −9.3 |
| Vitamin E | mg | 12 | 7.6 | 5.6 | −0.2 |
| Vitamin K | mcg | 120 | 93.2 | 45.7 | −170.4 |
| Zinc | mg | 9.4 | 4.8 | −0.3 | −6.8 |

TABLE 11
Amount of inadequacy of each “At Risk”
nutrient for 16:8 TRF for Females.

| Nutrient | Units | EAR/AI | 2.5% Gap | 50% Gap | 97.5% Gap |
|---|---|---|---|---|---|
| Calcium | mg | 880 | 573.7 | 77.8 | −1245.7 |
| Fiber | g | 23.4 | 12.2 | 1.8 | −8.1 |
| Food Folate | mcg | 320 | 202.0 | 1.3 | −179.0 |
| Magnesium | mg | 263 | 89.7 | −40.8 | −140.8 |
| Potassium | mg | 2300 | 63.0 | −1217.2 | −3369.2 |
| Vitamin A | mcg | 500 | 380.8 | −202.7 | −936.9 |
| Vitamin B12 | mcg | 2.0 | 1.6 | 0.0 | −4.1 |
| Vitamin D | mcg | 10 | 9.9 | 6.3 | −4.7 |
| Vitamin K | mcg | 90 | 71.1 | 10.0 | −241.4 |
| Zinc | mg | 6.8 | 3. | −0.6 | −4.5 |

The analysis of epidemiological data from NHANES database (Example 1) supports the fact the temporal eating patterns compatible with an intermittent fasting regimen are more likely to be associated with inadequate intakes of micronutrients. This suggest that the results produced by the diet simulator are consistent with patterns observed in the population. However, NHANES is essentially cross-sectional, while the diet simulator has a longitudinal dimensional that is difficult to measure in the population at a large scale.

### Recommendations (Applicable for Examples 1 and 2)

In order to construct dietary recommendations, we consider the upper and lower bounds of the intake recommendations to identify nutrient targets, without exceeding the UL (see FIGS. 1-4 for examples for calcium and folate). The “Amount of Inadequacy” is used to generate the dietary recommendations. First, we take the difference between the RDA, or the AI if no RDA is available, and the 2.5% intake percentile as the upper and the difference with the 97.5% intake percentile as the lower. All recommendations are based on an energy level for the diet of 2000 kcal. The upper end of the recommendation is capped to ensure that the recommendation does not exceed the UL.

We then employ a computerized Diet Simulator. By making certain assumptions, the Diet Simulator can provide a reasonable estimate of the nutritional gaps and thus provide recommendations on how to close those gaps from nutrients, foods, beverages, and/or dietary supplements, and their combinations, without requiring undue effort or input from the user. In this Example, assumptions included the following:
The individual eats a typical meal pattern (e.g., breakfast, lunch, dinner and one snack per day),
The individual eats typical food and beverage combinations for those meals and snacks (e.g., meals and snacks as reported in NHANES), and
The individual intends to follow a well-balanced diet in accordance with dietary recommendations (e.g., the USDA Healthy Eating Pattern).

The food composition database used by the Diet Simulator contains the nutrient content, including macro- and micro-nutrients for each food per 100 g and per representative serving sizes. In this example, the USDA Food Data Central databases are used to identify foods and beverages containing sufficient levels of the nutrients identified as “At Risk” in the preceding steps. The Diet Simulator is also linked to a recipe database so that recipes which contain food sources of the nutrients “At Risk” can be identified.

In both methodologies, we identified inadequacies (>80% inadequacy) in vitamin D, vitamin E and fiber, both at baseline diet and TRF diet. In both methodologies, we also identified inadequacies (>40% inadequacy) that are exclusively related to the TRF diet: calcium, folate, and zinc. In both methodologies, we also identified other inadequacies (>30% inadequacy) that are exclusively related to the TRF diet: magnesium and vitamin B12. Potassium, vitamin A and vitamin C inadequacies (>80%) were also identified in one of the methodologies (Example 1). We have selected only two of these nutrients to exemplify recommendations.

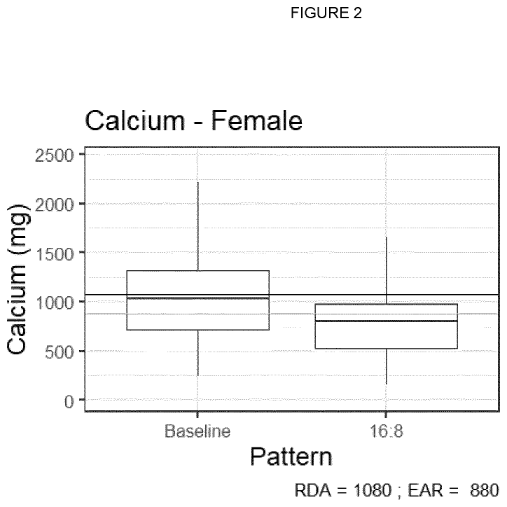

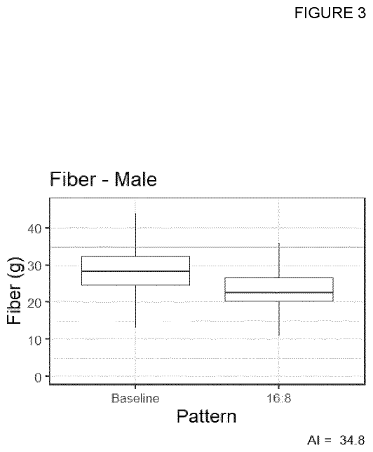

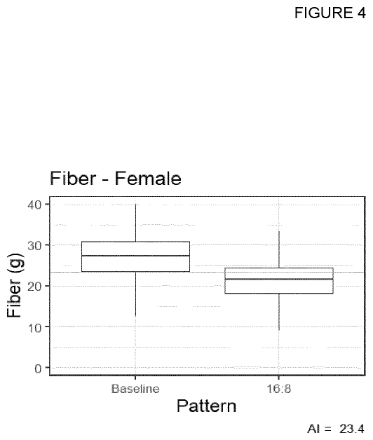

In these examples, fiber and calcium were identified as nutrients of need for the 16:8 TRF regimen. See FIGS. 1 to 4. FIG. 1 shows calcium intake ranges comparing simulated baseline intakes with 16:8 TRF regimen for males. FIG. 2 shows calcium intake ranges comparing simulated baseline intake with 16:8 TRF regimen for females. FIG. 3 shows Fiber intake ranges comparing simulated baseline intakes with 16:8 TRF regimen for males. FIG. 4 shows Fiber intake ranges comparing simulated baseline intakes with 16:8 TRF regimen for females.

Examples, such as the following dietary recommendations would be generated to accompany feedback on the TRF diets. In a similar manner, dietary recommendations for all of the nutrients identified as “At Risk” could be generated. As an illustration, we provide examples for calcium and dietary fiber as follows:

### Calcium:

Calcium could be recommended from food sources. For example, someone specifying a vegetarian diet may receive a recommendation for soybean curd (tofu) cubes; 1 cup (248 g) provides 275 mg of calcium. Similarly, a recipe for tofu with mixed vegetables, including broccoli and carrots with a soy-based sauce could be recommended; 2 cups (434 g) provides 286 mg of calcium.

Calcium could be recommended from beverages. For example, someone specifying a flexitarian diet may receive a recommendation for low-fat milk; 1 cup (246 g) of low-fat milk contains 310 mg of calcium.

Calcium could be recommended as a dietary supplement. Non-limiting examples of suitable forms of calcium include one or more calcium salts, such as calcium acetate, calcium carbonate, calcium chloride, calcium citrate, calcium gluconate, calcium lactate or mixtures thereof.

The amount of additional calcium recommended would depend on the needs identified in the simulations.

### Fiber:

Fiber could be recommended from food sources. For example, someone specifying a Vegan diet may receive a recommendation for cooked oatmeal; 1 cup (234 g) provides 4 mg of dietary fiber. Similarly, a recipe for oatmeal raisin cookies could be recommended; 2 cookies (48 g) provides 1.6 g of fiber.

Fiber could be recommended from dietary supplements. Non-limiting suitable forms of fiber supplements include those containing wheat dextrin, methylcellulose, psyllium husk, polycarbophil, guar fiber, glucomannan or P-glucans.

1. A computer implemented method or AI based system to identify and/or quantify individual nutritional inadequacies in an individual following or planning to follow a Time Restricted Feeding (TRF) regimen, comprising the steps of:
i) Identifying the individual's nutritional needs and/or dietary rules,
ii) Comparing the individual's nutritional needs and/or dietary rules with the nutrients provided by a TRF diet adapted, and
iii) Performing analyses to identify and quantify potential nutritional inadequacies.

2. A computer implemented method or system according to claim 1 wherein the Time Restricted Feeding regimen is selected from 16:8, 20:4, 12:12 protocol and variations thereof.

3. A computer implemented method or system according to claim 1, wherein the individual's nutritional needs are based on a characteristic selected from the group consisting of age, gender, height, weight, physical activity, lifestyle and medical condition.

4. A computer implemented method or system according to claim 1, wherein the dietary rules are based on individual's specific dietary restrictions or preferences.

5. A computer implemented method or system according to claim 1, wherein the TRF diet of step ii) is a simulated TRF diet.

6. A computer implemented method or AI-based system to mitigate nutrient inadequacies in an individual following or planning to follow a Time Restricted Feeding regimen, said method comprising:
i) Identifying nutritional inadequacies in the context of a defined TRF diet,
ii) Providing specific dietary recommendations and/or nutritional solutions to mitigate the identified nutritional inadequacies in said individual, and
iii) providing recommendations on lifestyle.

7. (canceled)

8. A computer implemented method or system according to claim 6, wherein said method or system comprises diet recommendations, menu recommendations and/or recipe recommendations.

9. A computer implemented method or AI-based system according to claim 6, wherein said method provides recommendations for food or nutrients selected from the group consisting of nutrients, foods and beverages, dietary supplements, combinations of foods and beverages as menu recommendations and/or recipe recommendations, and combinations thereof.

10. A method of mitigating nutrient inadequacies in an individual following or planning to follow a TRF regimen, said method comprising:
i) Identifying the individual's nutritional needs and/or dietary rules,
ii) Comparing the individual's nutritional needs and/or dietary rules with the nutrients provided by a TRY diet,
iii) Performing analyses to identify and quantify potential nutritional inadequacies,
iv) Providing specific dietary recommendations and/or nutritional solutions to mitigate the identified nutritional inadequacies in said individual, and
v) providing recommendations on lifestyle.

11-13. (canceled)
